(19) United States
(12) Patent Application Publication
(10) Pub. No.: US 2024/0167067 A1
(43) Pub. Date: May 23, 2024

(54) Cross-linkable xylans and methods of producing the same and their uses

(71) Applicant: CH-Bioforce Oy, Espoo (FI)

(72) Inventors: Lari Vähäsalo, Espoo (FI); Sebastian Von Schoultz, Espoo (FI); Nicholas Lax, Espoo (FI); Andrey Pranovich, Espoo (FI)

(21) Appl. No.: 18/283,472

(22) PCT Filed: Mar. 28, 2022
(86) PCT No.: PCT/FI2022/050196

§ 371 Date: Sep. 22, 2023

(30) Foreign Application Priority Data
Mar. 28, 2021 (FI) 20215354

(51) Int. Cl. C12P 19/04; C08J 3/24

(52) U.S. Cl. CPC C12P 19/04; C08J 3/24; C08J 2305/14

(57) ABSTRACT

The present invention relates to nanocrystalline xylan containing at least 0.1 free unsaturated groups per anhydroxylose unit, wherein free unsaturated groups are groups which exhibit unsaturated bonds, and which are capable of reacting with other groups, and the particle size of the xylan crystals is about 10 to 250 nm.

### TECHNICAL FIELD

The present invention relates to modified hemicelluloses and their production. In particular, the present invention concerns nanocrystalline xylan derivatives, methods of producing such derivatives, and uses thereof. The invention also concerns cross-linked nanocrystalline xylan polymers.

### BACKGROUND

Xylans are one of the most abundant polysaccharides in nature. The most abundant source of xylans are hardwood trees such as birch.

The backbone of xylans is made of β-1,4-linked xylose units. In nature, xylans are substituted with side groups, such as arabinose, 4-O-methyl-glucuronic acid and acetyl groups. These side units make xylans difficult to modify since some of the side groups can cause steric hindrance to accessing the hydroxyls in the xylose units. In addition, in alkaline media, acetyl groups are easily released from the polysaccharide which causes the pH to drop during substitution.

### OBJECTS AND SUMMARY

It is the aim of this invention to provide nanocrystalline xylan derivatives. It is in particular an aim to provide xylan derivatives that comprise free unsaturated groups, yet which are stable and can be substituted with other compounds. It is another aim of the invention to provide a method of modifying xylan to produce xylan derivatives. Such method should ideally be easy to control and economically interesting. It is a further aim of the invention to provide cross-linked xylan polymers, as well as to provide uses for such cross-linkable xylans.

According to a first aspect, the present description relates to nanocrystalline xylan containing at least 0.1 free unsaturated groups per anhydroxylose unit, wherein free unsaturated groups are groups which exhibit unsaturated bonds, and which are capable of reacting with other groups, and the particle size of the xylan crystals is about 10 to 250 nm.

According to another aspect, the present description relates to a method of modifying xylan comprising the steps of
providing xylan having side groups, wherein the xylan having side groups comprises nanocrystalline xylan, having a particle size of the xylan crystals of about 10 to 250 nm;
removing the side groups of the xylan to produce unsubstituted xylan having free hydroxy groups;
reacting the unsubstituted xylan with a reactant, which has a first functional group capable of reacting with the free hydroxy groups and a second functional group comprising free double bonds, to produce modified xylan; and
optionally recovering the modified xylan thus obtained.

According to a further aspect, the present description relates to a cross-linked xylan polymer obtainable by subjecting a nanocrystalline xylan as described above to cross-linking.

According to yet a another aspect, the present description relates to use of the present nanocrystalline xylan
as adhesives for medical or technical applications;
as thickeners in solvent solutions;
as lower-critical-solution-temperature materials; or
for forming transparent hydrogels or polymer films.

### BRIEF DESCRIPTION OF DRAWINGS

FIGS. 1A and 1B are photographs showing two vials of DS 1.3 allylated xylan at room temperature (FIG. 1A) and at 80° C. (FIG. 1B);

FIG. 2 shows storage modulus of allylated xylan with varying DS; and

FIG. 3 shows the storage modulus of DS 2.3 allylated xylan at varying temperatures.

### DETAILED DESCRIPTION

### Definitions

As used herein, the term “about” refers to a value, which is ±5% of the stated value. The term “essentially free”, unless otherwise states, means that there is at most 1% of the component the item is essentially free of. Any percentages that are not absolute percentages are weight-percentages.

Unless otherwise stated, properties that have been experimentally measured or determined herein have been measured or determined at room temperature.

Unless otherwise indicated, room temperature is 25° C. and body temperature is 37° C.

Unless otherwise stated, properties that have been experimentally measured or determined herein have been measured or determined at atmospheric pressure.

The expression “free unsaturated groups” stands for groups which exhibit unsaturated bonds, such as double or triple bonds, and which are capable of reacting with other groups, in particular with other groups of similar kind. In one embodiment, the groups with double or triple

“Side-groups” when used in connection with xylans stands in particular for groups which are coupled to the anhydroxylose backbone of the xylan molecule by ether bonds. Such bonds are obtained by the reaction of a hydroxy group on the anhydroxylose backbone with a reactive group present on another compound, such as an epoxy or glycidyl group.

The use of “a reactive group” also encompasses situations where there are more than one such reactive groups on the compound in question.

### DETAILED DESCRIPTION OF EMBODIMENTS

According to a first aspect, the present description relates to nanocrystalline xylan containing at least 0.1 free unsaturated groups per anhydroxylose unit, wherein free unsaturated groups are groups which exhibit unsaturated bonds, and which are capable of reacting with other groups, and the particle size of the xylan crystals is about 10 to 250 nm.

The nanocrystalline xylan as defined above has several beneficial properties. It is stable, yet can be substituted with other compounds, i.e. provides a basis for further processing in a variety of ways.

According to another aspect, the present description relates to a method of modifying xylan comprising the steps of
providing xylan having side groups, wherein the xylan having side groups comprises nanocrystalline xylan, having a particle size of the xylan crystals of about 10 to 250 nm;
removing the side groups of the xylan to produce unsubstituted xylan having free hydroxy groups;
reacting the unsubstituted xylan with a reactant, which has a first functional group capable of reacting with the free hydroxy groups and a second functional group comprising free double bonds, to produce modified xylan; and
optionally recovering the modified xylan thus obtained.

The present method is in particular useful for manufacturing the nanocrystalline xylan defined above. In the first step of the method, side group free xylans are produced. Such xylan polysaccharides are extremely stable and chemically resistant and can hence be substituted with a variety of chemicals, especially using reactants having a first reactive group comprising an epoxy functionality, such as epoxy or glycidyl group, and a second group having unsaturation, such as vinyl or allyl groups, to give the corresponding ethers upon reaction of the epoxy or glycidyl with the hydroxyl groups on the anhydroxylose units.

By reacting a compound containing a reactive group, such as epoxy, with water insoluble xylans, which do not contain side groups, xylan ethers are obtained which are water soluble substance. Such materials typically have properties of so-called lower-critical-solution-temperature (LCST) materials. The modified xylans are water soluble at low temperatures, but precipitates when heated. The temperature at which the material precipitates can be adjusted with the degree of substitution (D S) of the xylans.

Further, xylan derivatives with free allyl or vinyl groups, or similar groups containing a free double-bond, can be cross-linked. Cross-linking can be carried out chemically or with the aid of UV (ultra violet) light to form cross-linked polymers useful as, for example a binder or a hydrogel. The material can be cross-linked by cross-linking the xylans with each other or by cross-linking the xylans with other molecules, such as acrylates.

The present description thus relates also to a cross-linked xylan polymer obtainable by subjecting a nanocrystalline xylan as described above to cross-linking.

According to yet a another aspect, the present description relates to use of the present nanocrystalline xylan
as adhesives for medical or technical applications;
as thickeners in solvent solutions;
as lower-critical-solution-temperature materials; or
for forming transparent hydrogels or polymer films.

The present materials are thus useful in a variety of applications, such as in the use as an adhesive in medical applications. The material can be applied as a liquid for example at room temperature. Since it precipitates at body temperature, it can be employed subjected to cross-linking to form an adhesive. Further, the xylans can be used as adhesive thickeners, films and rheology modifiers.

In an embodiment, the natural (or native) side groups, typically attached by ether bonds to the anhydroxylose backbone, if any, present in the xylans, are first removed. Examples of native side groups which are removed include arabinose, uronic acid and acetyl groups and combinations thereof.

The pure xylan polysaccharides thus obtained are substituted with new side groups with a free double bond, which can react with double bonds of other xylans and hence cross-link a material comprising such xylan polymers. Examples of such groups include allyl and vinyl groups.

The xylans obtained by peeling-off of side groups which they conventionally contain, are also referred to as “pure” xylans. This expression refers to xylan molecules, which comprise, or consist of or consist essentially of an anhydroxylose chain. Such an anhydroxylose chain exhibits hydroxy groups, typically at e.g. positions 2 and/or 3 and/or 5, some of which can be dangling (i.e. the hydroxyl group is linked typically by an alkylene, in particular methylene, linker to the anhydroxylose chain), whereas others can be bonded directly to the anhydroxylose chain.

Natural xylan polysaccharides contain side groups, such as acetyl, which prevents effective substitution of the xylan backbone with reactive side groups such as allyl and vinyl groups. In embodiments of the present technology, at least a majority, in particular all, of the side groups are removed.

The xylan starting material is preferably crystalline xylan, such as nanocrystalline xylan. The particle size of the xylan crystals is about 10 to 250 nm, in particular 30 to 100 nm. Particle size was determined by transmission electron microscopy (TEM) by taking 1-10 TEM-photos of the material, measuring the largest diameter of 5-10 particles in each photo and calculating the number average (arithmetic mean) of the measurements, leading to the particle size of the material.

The starting xylan typically has a degree of polymerisation of 4 to 600, in particular 8 to 300.

In one embodiment, side groups are removed from the xylan starting material by subjecting it to an alkaline media, for example by heating it in aqueous alkaline media, and optionally in the presence of a reducing agent.

In one embodiment, the side groups are removed by contacting xylan in an aqueous medium with an enzyme for peeling off the side groups.

Thus, in one embodiment, most or all of the side groups are removed by heating xylan based starting material in an alkaline media and/or reducing environment, such as in the presence of NaBH₄ or other reducing agents. In one embodiment, enzymes, in particular enzymes capable of peeling off the side groups from the xylan backbone, are used either alone or in conjunction with another treatment for removing side groups from the xylan starting material.

In one embodiment, side groups are removed by
a) heating xylan in alkaline media and optionally reducing environment; or
b) contacting xylan in an aqueous medium with an enzyme for peeling off of the side groups; or
c) a combination of steps a and b in any order.

In one embodiment, during the reaction wherein the side groups natively present on the xylan are removed, the xylan material is heated in an aqueous medium to a temperature of about 50 to 100° C., such as 60 to 100° C. at ambient pressure. Examples of such groups include arabinose, uronic acid (e.g. glucuronic acid, such as 4-methyl glucuronic acid, or hexenuronic acid) and acetyl groups

If operating at alkaline conditions, the pH is typically 9 to 14, in particular 10 to 14. The reaction time is 0.1 to 6 hours, typically about 0.2 to 4 hours.

In one embodiment, enzymatic treatment is carried out at temperatures of about 20 to 65° C. and a pH of about 5 to 9, in particular 6 to 8. The reaction time is 0.1 to 12 hours, typically about 0.2 to 6 hours.

In one embodiment, a xylan is provided which is essentially free from side groups selected from arabinose, uronic acid and acetyl groups and combinations thereof. Typically, there is less than 0.2, in particular less than 0.1, for example less than 0.05 or less than 0.01 such native side groups per anhydroxylose unit.

One embodiment comprises providing, for example by the above-described procedure, unsubstituted xylan having free hydroxy groups exhibiting per anhydroxylose unit less than 0.1, in particular less than 0.05, such as less than 0.01 side groups, in particular selected from the group of acetyl groups.

The pure, side group free, xylan is stable and has a long shelf life of, for example, up to 360 days.

It has been found that pure xylan polysaccharides are readily substituted with reactive groups. Substitution can be carried out such that a predetermined result (in particular a predetermined degree of substitution) can be obtained.

Thus, preferably, in the next step of the method, the unsubstituted xylan is reacted with a reactant containing a first reactive group selected from epoxy and glycidyl groups. The epoxy or glycidyl groups will react with hydroxyl groups on the anhydroxylose backbone and form an ether bond.

The reactant typically contains a second reactive group, spaced apart from the first reactive group, capable of introducing double bonds into the reaction product of the reaction between the unsubstituted xylan and the reactant.

Examples of reactants include glycidyl and epoxy ether comprising an ether group that contains a hydrocarbon radical with at least one unsaturation. Typically, the unsaturation consist of at least one double or triple bond.

In one embodiment, the glycidyl or expoxy ether comprises a 2 to 10 carbon hydrocarbon residue with 1 to 3 double or triple bonds, in particular 2 to 3 carbon atoms and a double bond at the terminus of the hydrocarbon radical. In one embodiment, in addition to the first reactive group, the reactant contains a second reactive group selected from allyl and vinyl groups and combinations thereof.

In one embodiment, vinyl and allyl glycidyl ethers and combinations thereof are used as reactants.

In one embodiment, the unsubstituted xylan is reacted with the reactant at a molar ratio of reactant to anhydroxylose units at 100:1 to 1:1. Typically, there is a molar excess of reactant in relation to available hydroxyl functionalities on the anhydroxylose; thus the molar ratio of reactant to anhydroxylose units is preferably about 100:1 to about 10:1.

In one embodiment, the reaction is carried out in aqueous medium at a pH in the range of 6 to 14, for example 7 to 12.

In one embodiment, the reaction between the unsubstituted xylan and the reactant is carried out in an aqueous medium and preferably at ambient pressure, though it is possible to operate at reduced pressure (vacuum), such as at 10 to 900 mbar (abs) or excess pressure, for example at 1.1 to 15 bar (abs).

The reaction temperature of the reaction between the unsubstituted xylan and the reactant is typically, in particular when operating at ambient pressure, about 20 to 100° C., such as 25 to 100° C., for example 30 to 100° C. or 50 to 100° C. In one preferred embodiment, the reaction is carried out at reflux conditions.

The reaction duration is about 0.1 to 10 hours, in particular about 10 minutes to 5 hours.

Upon completion of the reaction between the unsubstituted xylan with vinyl or allylglycidyl ether in aqueous medium, the pH of the reaction mixture is, in one embodiment, adjusted to a value in the range of about 4 to 7 in particular 5 to 6.5, such as 5.5 to 6.

The pH can be adjusted with an acid, in particular an aqueous acid, such as an organic or a mineral acid. In one embodiment, a carboxylic acid is employed; examples include alkanoic acids, such as formic acid.

The work-up of the reaction mixture comprising modified xylan typically includes subjecting the modified xylan to purification and optionally other post-treatment steps. In particular, salts and other side-products are typically removed.

In one embodiment, the modified xylan is subjected to dialysis, for example by membrane filtration.

As a result of the reaction, xylan containing at least 0.1 free unsaturated groups per anhydroxylose unit is obtained. In particular xylan is provided, in which there are 0.3 to 2.5 unsaturated groups per anhydroxylose unit. Such unsaturated groups are reactive groups.

According to an embodiment, the free unsaturated groups of the xylan are allyl or vinyl groups or combinations thereof. Such groups can be achieved for example by using allyl of vinyl ethers as reactant.

The modified xylan is also essentially free from side groups selected from arabinose, uronic acid and acetyl groups and combinations thereof, in particular when operating the above-disclosed method in which they are removed from the starting material before reaction with the reactants.

In one embodiment, the modified xylan has a degree of polymerisation of 4 to 200, in particular 8 to 150. The degree of polymerisation can be for example from 4, 5, 6, 7, 8, 9, 10, 15, 20, 25, 30, 40, 50, 60, 70, 80, 90, 100, 120, 150 or 170 up to 7, 8, 9, 10, 15, 20, 25, 30, 40, 50, 60, 70, 80, 90, 100, 120, 150, 170, 180, 190 or 200.

In one embodiment, wherein the reactant is allylglycidyl ether, modified xylan is obtained which is generally water-soluble. In addition, it has been found that such material becomes a so-called lower-critical-solution-temperature (LCST) material, in one embodiment having a lower-critical-solution-temperature of 90° C. or less. This particular embodiment would thus allow manufacturing of LCST material not previously known, and with a new manufacturing method.

As a result, the modified xylans are water-soluble at ambient temperatures or even lower, but they precipitate when heated. The temperature, at which the material precipitates, can be controlled by adjusting the degree of substitution (D S) of the xylans.

In one embodiment, the modified xylan exhibits a degree of substitution of allyl groups in the range from 1.0 to 2.3 per anhydroxylose unit and a lower-critical-solution-temperature of no more than 80° C., preferably 70° C. or less, in particular than 60° C. or less.

According to an embodiment, the nanocrystalline xylan exhibits solubility in water at temperatures of 0 to 10° C., at ambient pressure. This is achieved in particular when using vinyl or allylglycidyl ether.

The present materials are attractive for a variety of applications, such as in the use as an adhesive in medical applications. The material can be applied for example at room temperature whereby it precipitates at body temperature as an adhesive which then can be cross-linked to form an extremely good adhesive.

In one embodiment, xylan or xylan containing composition is applied at 10 to 30° C., in particular about room temperature, on or onto an object (or surface of an object) and allowed to precipitate at a higher temperature, such as body temperature, to achieve solidification of the xylan or xylan containing material, which is subsequently cross-linked to form an adhesive.

According to an embodiment, cross-linking is carried out in the presence of an initiator, such as a UV initiator, or in the presence of radicals, such as radicals obtained from a peroxide compound. In one embodiment, the modified xylans are thus cross-linked using UV light or with light having a greater wavelength. Photo initiators can be added in order to increase the speed of crosslinking between the reactive groups. Also peroxide-based cross-linking can be employed.

In one embodiment, the materials are used as adhesives for medical or technical applications. Here, the functionalised xylans are first mixed with different other adhesives especially those, in the form of monomers, oligomers or polymers, with reactive carbon-carbon double bonds, such as acrylates or metacrylates. Then the xylan is subjected to cross-linking in the presence of the other compounds containing reactive carbon-carbon double bonds.

The cross-linked materials, in particular adhesives, can contain filler particles, such as silica or other inorganic particles. They may be incorporated into the material by carrying out the cross-linking in their presence.

Yet in another embodiment, the materials are used as a thickener in solvent solutions either so that the functionalised xylans are cross-linked first, then mixed with a preferred material such as pigments or that the solution or dispersion is cross-linked after the materials have been mixed.

Yet in another embodiment, the materials are used as a lower-critical-solution-temperature material, where the xylans are precipitated from the solvent by increasing the temperature of the solvent.

Yet in another embodiment, the materials are used for forming transparent hydrogels or polymer films after drying.

Due to the much lower molar mass of xylans compared to cellulose and starch, xylans can be derivatised to produce water-soluble materials. The solubility of xylans solvents which are unipolar than water can be adjusted by substituting xylans with non-polar side groups such as methyl groups.

In adhesives, cross-linking is an attractive property as the material can be cured to form a strong adhesive. For example, dental fillings are often acrylic based which, with the aid of photo initiators and UV-light, can be cross-linked to form a strong filling.

Thus, as explained above, in embodiments, xylan nanocrystals are functionalised using allyl glycidyl ether or vinyl glycidyl ether in order to introduce allyl or allyl groups with reactive carbon-carbon double bonds located at the end position of the reactive group. The formed molecules can be cross-linked using UV light or with light having a greater wavelength. Photo initiators can be added in order to increase the speed of crosslinking between the reactive groups. Also other crosslinking methods can be used, such as the use of peroxides and other substances capable of releasing radicals.

The following non-limiting examples illustrate embodiments.

### EXAMPLES

Xylans nano crystals (XNC) were derivatised and the functionalised materials were tested as will be discussed in the following examples.

### Example 1

16 g D.I. water was put into a 100 mL three-necked round flask equipped with a magnet bar and 4.02 g nanocrystalline xylan (XNC) slowly added (corresponding to 30 mmol anhydroxylose (AXU) units, under intensive stirring at ambient conditions. The reflux condenser was connected to the flask, the temperature of the oil bath was increased to 65° C. and the obtained 20% suspension was stirred for 1 hour.

3.15 mL of 40% NaOH, which corresponds to 45 mmol NaOH (45×40 mg=1800 mg NaOH, contained in 4500 mg or in 3.15 mL of 40% NaOH (ρ=1.422 g/cm³)) were added into the flask under stirring; the temperature was raised to 85° C. and the content was mixed for the next 1 hour.

10.58 mL allylglycidyl ether (AGE), which corresponds to 90 mmol AGE (90×114=10.26 g or 10.58 mL (ρ=0.97 g/cm 3)), were added into the flask, the temperature was raised up to 85° C. and the content was mixed during a certain time to reach the desired DS (Table 1).

A similar protocol was used for XNC modification to the product with DS 2.3, except using a higher AGE dose, i.e. 210 mmol, which corresponds to 24.68 mL AGE. The reference XNC sample was obtained from the same protocol, but with no AGE addition was made (blank treatment).

The flask was cooled down and the content was acidified with ca 106 droplets of 50% formic acid to pH 5.6-5.8.

To purify the modified XNC products from salts and other low-molar mass chemicals and side-products, they were subjected to dialysis using a membrane tube (diameter 28.6 mm, MWCO 12-14 kDa). The conductivity of the last washing waters was in the range of 2.0-2.4 μS/cm.

The preceding steps were repeated with various amounts of AGE. The results are summarised in Table 1. In Table 1, * stands to indicate that the mmoL (or mmol) amounts were calculated and that 1 mmol of AXU equated to 134 mg, while ** indicates similary that 1 mmol of AGE equated to 114 mg.

TABLE 1
Different modifications of allylglycidyl ether (AGE)

| XNC as | 40% | | Temper- | | |
|---|---|---|---|---|---|
| anhydroxylose units | NaOH, | AGE, ** | ature, | Time, | Expected |
| (AXU), * mmoL | mL | mmoL | ° C. | hours | DS |
| 30 | 45 | — | 85 | 24 | — |
| 30 | 45 | 90 | 85 | 4.5 | 0.3 |
| 30 | 45 | 90 | 85 | 8 | 0.6 |
| 30 | 45 | 90 | 85 | 24 | 1.3 |
| 30 | 45 | 210 | 85 | 24 | 2.3 |

### Example 2

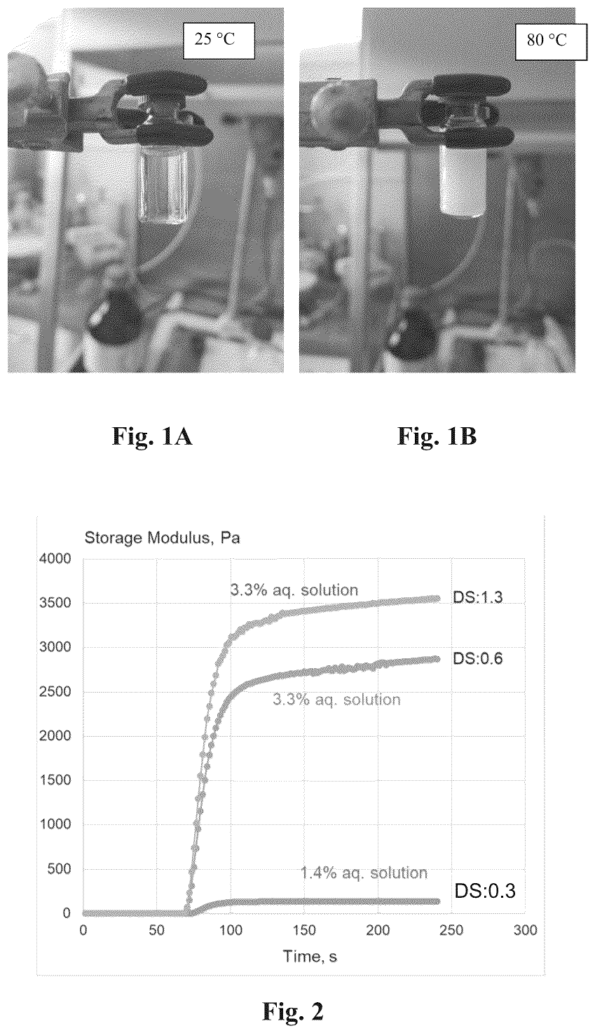

In this experiment, the temperature dependency of the different products with varying degree of substitution was measured. It was noted that some materials were insoluble in water and others were completely soluble. The lower-critical-solution-temperature of DS 0.3 was above 100° C. (actual temperature not measured), for DS 0.6 precipitation started at 90° C., for DS 1.3 precipitation started at 50-60° C. and was completely precipitated at 80° C. (FIG. 1), for DS 2.3 precipitation started at 15° C. and was complete at 25° C. The experiment shows that the lower-critical-solution-temperature can freely be adjusted with the degree of substitution.

### Example 3

In this experiment, the film formation capability of the various derivatised xylans was measured. A water or acetone solution of the materials was applied on a Teflon surface and the solutions were irradiated with 365 nm UV light. Without an UV-initiator in the solutions the polymerization speed was very low, especially the acetone solutions dried into an opaque film before the polymerization had taken place. However, by adding lithium phenyl-2,4,6-trimethylbenzoyl phosphinate (LAP) to the solutions complete polymerization was achieved and a transparent gel had been formed in under 2 minutes. After the solvent had evaporated, a transparent ridged film had formed on the Teflon plate.

### Example 4

In this experiment, the storage moduli of water solutions were measured, having DS 0.3, DS 0.6 and 1.3 allylated xylan with UV-initiator added. The UV-light irradiation started 60 sec after the beginning of the measurement and the irradiation continued for 180 sec.

The results are shown in FIG. 2.

As will appear from FIG. 2, the number of reactive groups affect how viscose the material becomes during cross-linking. The amount of UV-initiator LAP had also a large effect on the final storage modulus of the material. For DS 1.3 allylated xylan the storage modulus was 100, 3500 and 17000 for LAP dose of 2.5, 5 and 10 mg/mL resp.

In order to test the temperature dependency, the storage modulus of DS 2.3 allylated xylan was tested.

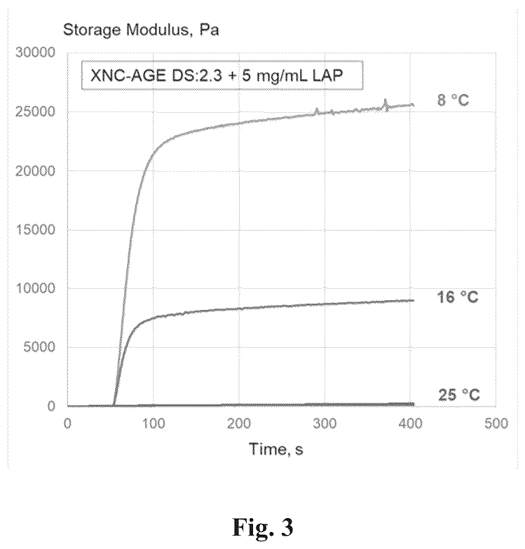

The results are shown in FIG. 3 where it can be seen that the material which is water soluble in refrigerator temperature and precipitates at room temperature has a much greater storage modulus at the temperature where they are soluble prior to cross-linking.

### Example 5

Vinyl glycidyl ether was used to perform the same derivatisation as with allyl glycidyl ether.

It was found that the material also exhibited the same LC ST property as the allylated xylans and formed transparent films when cross-linked.

Thus, as the example show, biopolymers with same type of properties as described for the allylated xylans are obtained with vinyl ethers.

It is to be understood that the embodiments disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and examples may be referred to herein along with alternatives for the various components thereof.

It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the description, numerous specific details are provided, such as examples of lengths, widths, shapes, etc., to provide a thorough understanding of embodiments. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

The described method for producing derivatised pure xylans, starting from pure xylans without the normal side-groups in plant xylans, produces new high performance biopolymers for many applications. This lower-critical-solution-property seen for the present xylans give rise to interesting applications, as well as the modified xylans' ability to form cross-links and cross-linked materials. The new derivatives find uses in various technical and medical fields, in particular for producing coatings, films and adhesives in particular as substitutes for similar fossil-derived materials.

### Abbreviations

AGE stands for allylglycidyl ether
AXU stands for anhydroxylose unit
DP stands for degree of polymerisation
DS stands for degree of substitution
LAP stands for Lithium phenyl-2,4,6-trimethylbenzoyl phosphinate
LCST stands for lower-critical-solution-temperature
XNC stands for xylan nanocrystals

The project leading to this application has received funding from the European Union's Horizon 2020 research and innovation programme under grant agreement No 848596.

1. Nanocrystalline xylan comprising at least 0.1 free unsaturated groups per anhydroxylose unit, wherein the free unsaturated groups are groups which exhibit unsaturated bonds, and which are capable of reacting with other groups, and wherein the particle size of the xylan crystals is about 10 to 250 nm.

2. Nanocrystalline xylan according to claim 1, wherein there are 0.3 to 2.5 unsaturated groups per anhydroxylose unit.

3. Nanocrystalline xylan according to claim 1, wherein the free unsaturated groups comprise allyl groups, vinyl groups, or combinations thereof.

4. Nanocrystalline xylan according to claim 1, wherein the nanocrystalline xylan is essentially free from side groups selected from the group consisting of arabinose, uronic acid, acetyl groups, and combinations thereof.

5. Nanocrystalline xylan according to claim 1, wherein the nanocrystalline xylan has a degree of polymerization of 4 to 200.

6. Nanocrystalline xylan according to claim 1, wherein the particle size of the xylan crystals is 30 to 100 nm.

7. Nanocrystalline xylan according to claim 1, wherein the nanocrystalline xylan is comprises a lower-critical-solution-temperature (LCST) material having a solution temperature of 90° C. or less.

8. Nanocrystalline xylan according to claim 1, wherein the nanocrystalline xylan has a degree of substitution of allyl groups in the range from 1.0 to 2.3 per anhydroxylose unit and a lower-critical-solution-temperature of no more than 80° C.

9. Nanocrystalline xylan according to claim 1, exhibiting solubility in water at temperatures of 0 to 10° C., at ambient pressure.

10. A method of modifying xylan comprising the steps of:
providing xylan having side groups, wherein the xylan having side groups comprises nanocrystalline xylan, having a particle size of the xylan crystals of about 10 to 250 nm;
removing the side groups of the xylan to produce unsubstituted xylan having free hydroxy groups;
reacting the unsubstituted xylan with a reactant, which has a first functional group capable of reacting with the free hydroxy groups and a second functional group comprising free double bonds, to produce modified xylan; and
optionally recovering the modified xylan thus obtained.

11. The method according to claim 10, wherein side groups are removed:
a) by heating xylan in alkaline media and optionally reducing environment;
b) by contacting xylan in an aqueous medium with an enzyme for peeling off of the side groups; or
c) by a combination of steps (a) and (b) in any order.

12. The method according to claim 10 or 11, wherein the unsubstituted xylan having free hydroxy groups exhibits per anhydroxylose unit less than 0.1.

13. The method according to claim 10, wherein unsubstituted xylan is reacted with a reactant containing a first reactive group selected from the group consisting of epoxy and glycidyl groups.

14. The method according to claim 10, wherein the reactant contains a second reactive group selected from the group consisting of allyl groups, vinyl groups, and combinations thereof.

15. The method according to claim 10, further comprising reacting the unsubstituted xylan with the reactant at a molar ratio of anhydroxylose units to reactant at 1:100 to 1:10.

16. The method according to claim 10, wherein the particle size of the xylan crystals is 30 to 100 nm.

17. The method according to claim 10, further comprising the step of subjecting the modified xylan to membrane filtration.

18. A cross-linked xylan polymer obtainable by subjecting nanocrystalline xylan according to claim 1 to cross-linking.

19. The cross-linked xylan polymer according to claim 18, wherein the xylan is subjected to cross-linking in the presence of other compounds containing reactive carbon-carbon double bonds.

20. The cross-linked xylan polymer according to claim 19, wherein the other compounds are selected from the group consisting of monomers, oligomers, and polymers having reactive carbon-carbon double bonds.

21-24. (canceled)
